(19) United States
(12) Patent Application Publication
(10) Pub. No.: US 2025/0250387 A1
(43) Pub. Date: Aug. 7, 2025

(54) Method For Making A Polyurethane Carrier Composite Immobilized Substance

(71) Applicant: Sz-Chwun John Hwang, Hsinchu City (TW)

(72) Inventors: Ming Chiun Lee, Hsinchu City (TW); Sz-Chwun John Hwang, Hsinchu City (TW)

(21) Appl. No.: 19/047,675

(22) Filed: Feb. 7, 2025

Related U.S. Application Data
(60) Provisional application No. 63/550,628, filed on Feb. 7, 2024.

(51) Int. Cl. C08G 18/12; C08G 18/08; C08G 18/48; C08G 101/00

(52) U.S. Cl. CPC C08G 18/12; C08G 18/14; C08G 18/48; C08G 2101/00; C08G 2110/0025; C08G 2110/0083

(57) ABSTRACT

A method for making a polyurethane carrier composite immobilized substance is provided, and the method includes: mixing a polyol, an immobilized substance and water to form a first mixture; and mixing an isocyanate with the first mixture to form a second mixture. The second mixture undergoes a foaming reaction, and a polyurethane carrier entrapping the immobilized substance is formed, where the weight of water accounts for 20% to 50% of the weight of the polyol and the water, and the weight ratio of the isocyanate to a sum of the polyol and the water is between 1:2 and 2:1, the weight ratio of the isocyanate to the polyol is between 1:2 and 4:1, and the weight ratio of the isocyanate to the water is between 1:2 and 6:1.

### CROSS-REFERENCE TO RELATED APPLICATION

This application claims the benefit of U.S. Provisional Application No. 63/550,628, filed on Feb. 7, 2024. The entire contents of the above-identified patent application are incorporated herein by reference.

### FIELD OF THE INVENTION

The present invention relates to a method for making a polyurethane carrier composite immobilized substance, and particularly to a method for making a polyurethane carrier entrapping an immobilized substance.

### BACKGROUND OF THE INVENTION

The earliest material used in the technological development of entrapping beads in U.S. Pat. No. 4,400,391 (1983) is natural polysaccharides such as algin (simply referred to as ALG). Any one of the ions such as calcium, barium, lead, copper, strontium, cadmium, zinc, nickel, aluminum, or a combination thereof is used as the core metal to form algin complexes. Since the above algin complexes are mostly granular, they are hereinafter simply referred to as algin beads. The formation of algin beads is very rapid (the time for formation requires 0.1-25 minutes, and 1-10 minutes is sufficient). In the manufacturing process, when a solution containing algin (with an algin concentration of 0.5-3%, preferably 1-1.5%) is dropped into a forming solution containing the above metal ions (with a concentration of at most 50%, preferably 1-15%) using a dropwise device, the algin beads can be formed instantaneously. Other patent applications developed based on the above U.S. patent all use a solution containing calcium ions as the forming solution. However, relatively, calcium and phosphates can also rapidly form calcium phosphate precipitates (Ksp<2.07×10⁻³³). As a result, the algin beads are prone to collapse because calcium is snatched away by the phosphates commonly present in the external environment, causing the algin beads to disintegrate within just 1-3 months.

It is known that algin beads made of natural materials are prone to collapse, and then the polyvinyl alcohol (PVA) beads invented by Professor Chen Kuo-cheng (U.S. Pat. No. 5,290,693, 1994) came out, where the polyvinyl alcohol beads are formed by using a polyvinyl alcohol-boric acid method (PVA-boric acid method). However, this forming method is not as easy to form as algin beads. Unless PVA materials with a degree of alkalization (also referred to as the degree of saponification) exceeding 99.9% or even higher are used, the beads can be formed rapidly in the boric acid solution. However, PVA with such a high degree of alkalization cannot be mass-produced. As a result, almost all PVA beads on the market add algin to help shape (Mainland China Published Patent No. 107937381A and Mainland China Published Patent No. 110078206A). Alternatively, algin is first used for rapid forming of PVA beads, and then the PVA beads undergo polyacetal reaction to make the structure of the PVA beads more robust (Republic of China Invention Patent No. I579035). However, these combinations of artificial plastics (PVA) and natural colloids (algin) do not remain stable for a long time just because of the addition of artificial plastics. Artificial plastic beads that lose natural polysaccharides are full of pores, and their lifespan is not too long. In addition, beads made of PVA, algin, or PVA/algin all share a common drawback. That is, during denitrification, anaerobic fermentation, and the removal of hydrogen peroxide by enzymes, the beads will expand and even burst due to the generation of a large amount of gas.

Republic of China Patent No. I425050 (2010) proposes polyurethane (PU)/algin (PU/ALG) beads. Although algin is also used to assist in the forming of PU, the final polymerization of PU does not rely on crosslinking but on the instability of the physical properties of PU, causing it to curl up into a mass on its own. Since the condition for forming beads by leveraging the instability of PU needs to be carried out at a very high PU content (greater than or equal to 30%). Since the price of PU is far more expensive than that of other artificial plastics, it is not feasible in terms of economic benefits. On the other hand, this mechanism of using phase change and self-aggregation of pure substances makes it difficult for the beads to form pores that can accommodate the entrapped substances.

Therefore, providing a bead or carrier, which is easy to form, has good durability, can avoid expansion or rupture, and has an immobilized substance with good activity, is an urgent problem that relevant industry practitioners are eager to solve.

### SUMMARY OF THE INVENTION

The present invention provides a method for making a polyurethane carrier composite immobilized substance. This method can produce a polyurethane carrier with an immobilized substance with good activity, which has the advantages of being easy to form, having good durability, and being able to avoid expansion or rupture.

In order to achieve one or some or all of the above objectives or other objectives, an embodiment of the present invention provides a method for making a polyurethane carrier composite immobilized substance, including: mixing a polyol, an immobilized substance and water to form a first mixture; and mixing an isocyanate with the first mixture to form a second mixture, and subjecting the second mixture to a foaming reaction to form a polyurethane carrier entrapping the immobilized substance, where a weight of the water accounts for 20% to 50% of a total weight of the polyol and the water, a weight ratio of the isocyanate to a sum of the polyol and the water is between 1:2 and 2:1, a weight ratio of the isocyanate to the polyol is between 1:2 and 4:1, and a weight ratio of the isocyanate to the water is between 1:2 and 6:1.

In an embodiment of the present invention, above after the step of mixing the isocyanate with the first mixture to form the second mixture, further includes: pouring the second mixture into a mold.

In an embodiment of the present invention, the above polyol includes a polyether polyol.

In an embodiment of the present invention, the above polyol has an OH value between 80 and 300.

In an embodiment of the present invention, the above isocyanate includes at least one of methylenediphenyl diisocyanate (MDI) and a prepolymer of methylenediphenyl diisocyanate.

In an embodiment of the present invention, the above isocyanate has an NCO value between 20 and 32.

In an embodiment of the present invention, the above immobilized substance includes at least one of a microbial cell, an enzyme, activated carbon, a catalyst and sludge.

In an embodiment of the present invention, the above first mixture further includes an auxiliary agent, and the auxiliary agent includes a catalyst and/or a foam stabilizer.

In an embodiment of the present invention, the above catalyst includes at least one of dimethylamino cyclohexane, dibutyltin dilaurate and bismuth octoate.

In an embodiment of the present invention, a weight of the above catalyst accounts for 100 ppm to 500 ppm of a weight of the second mixture.

In an embodiment of the present invention, the above foam stabilizer includes a silicone surfactant.

In an embodiment of the present invention, a weight of the foam stabilizer accounts for 0.1% to 10% of a weight of the second mixture.

Since the polyol, the immobilized substance, the isocyanate and the water are adopted in the present invention, the polyurethane carrier composite immobilized substance, which is easy to form, has good durability and can avoid expansion or rupture, can be produced. In addition, in the present invention, since the weight of the water accounts for 20% to 50% of the total weight of the polyol and the water, and the weight ratio of the isocyanate to the sum of the polyol and the water is set to be between 1:2 and 2:1, the weight ratio of the isocyanate to the polyol is between 1:2 and 4:1, and the weight ratio of the isocyanate to the water is between 1:2 and 6:1, the temperature of the foaming reaction of the polyurethane carrier can be maintained below 70° C., which helps the immobilized substance entrapped in the polyurethane carrier to have good activity.

In order to make the above and other objectives, features and advantages of the present invention more clearly understood, embodiments are given below and described in detail with reference to the accompanying drawings.

### BRIEF DESCRIPTION OF THE DRAWINGS

FIG. 1 is a schematic flow diagram of a method for making a polyurethane carrier composite immobilized substance according to an embodiment of the present invention.

FIG. 2 shows change of ammonia nitrogen in water over time in a fed-batch shake-flask experiment in a 1-L serum bottle using a polyurethane carrier (200 mL) entrapping Microbe® strain according to an embodiment of the present invention.

FIG. 3 shows change of ammonia nitrogen in water over time when the polyurethane carrier used in FIG. 1 is placed in a 12-L (20 cm×20 cm×30 cm) fish tank to perform a fed-batch shake-flask experiment according to an embodiment of the present invention.

FIG. 4 shows change of ammonia nitrogen in water over time in a fed-batch shake-flask experiment in a 1-L serum bottle using a 300 mL polyurethane carrier entrapping Microbe® strain according to an embodiment of the present invention.

FIG. 5 shows change of ammonia nitrogen in water over time in a fed-batch shake-flask experiment in a 1-L serum bottle using a 600 mL polyurethane carrier entrapping Microbe® strain according to an embodiment of the present invention.

FIG. 6 shows change of ammonia nitrogen in water over time in a fed-batch shake-flask experiment in a 1-L serum bottle using a 200 mL polyurethane carrier entrapping Yusbio nitrifying strain according to an embodiment of the present invention.

FIG. 7 shows change of nitrate nitrogen in water over time in a fed-batch shake-flask experiment in a 1-L serum bottle using a 200 mL polyurethane carrier entrapping Azoo® denitrifying strain according to an embodiment of the present invention.

FIG. 8 and FIG. 9 show change of ammonia nitrogen in water over time in a fed-batch shake-flask experiment in a 1-L serum bottle using a 200 mL polyurethane carrier entrapping sludge according to an embodiment of the present invention. FIG. 8 shows experimental data from day 1 to day 14; and FIG. 9 shows experimental data from day 15 to day 35.

FIG. 10 shows a state of a polyurethane carrier entrapping sludge undergoing anaerobic fermentation according to an embodiment of the present invention.

FIG. 11 shows that the same bacterial source as in FIG. 10 is used for denitrification purposes and exhibits a black anaerobic state after standing for a long time according to an embodiment of the present invention.

FIG. 12 shows a polyurethane carrier entrapping an immobilized substance including a catalyst and formed by foaming in a paper cup according to an embodiment of the present invention.

FIG. 13 shows change of H₂O₂ in water over time using the polyurethane carrier entrapping the catalyst shown in FIG. 12 for treating simulated wastewater with 4,000 mg/L H₂O₂ according to an embodiment of the present invention.

FIG. 14 shows sampling detection of a hydrogen peroxide residual rate in the 7th to 13th tests according to an embodiment of the present invention.

FIG. 15 shows the hydrogen peroxide removal efficiency of a polyurethane carrier at a fixed reaction time (90 minutes) according to an embodiment of the present invention.

FIG. 16 is a photograph of a PVC column used in an experiment of delaying enzyme loss of a polyurethane carrier according to an embodiment of the present invention.

FIG. 17 is a photograph of a polyurethane carrier entrapping multiple PVA beads according to an embodiment of the present invention.

FIG. 18 is a photograph of a spherical object immersed in an aqueous solution according to an embodiment of the present invention.

FIG. 19 is a photograph of a spherical object and a New Taiwan Dollar fifty-dollar coin according to an embodiment of the present invention.

FIG. 20 is a schematic cross-sectional view of a spherical object including a polyurethane carrier inside according to an embodiment of the present invention.

FIG. 21 shows a three-dimensional view of a polyurethane carrier suitable for purifying natural water bodies according to an embodiment of the present invention.

FIG. 22 shows a schematic bottom view of the polyurethane carrier in FIG. 21.

### DETAILED DESCRIPTION OF PREFERRED EMBODIMENTS

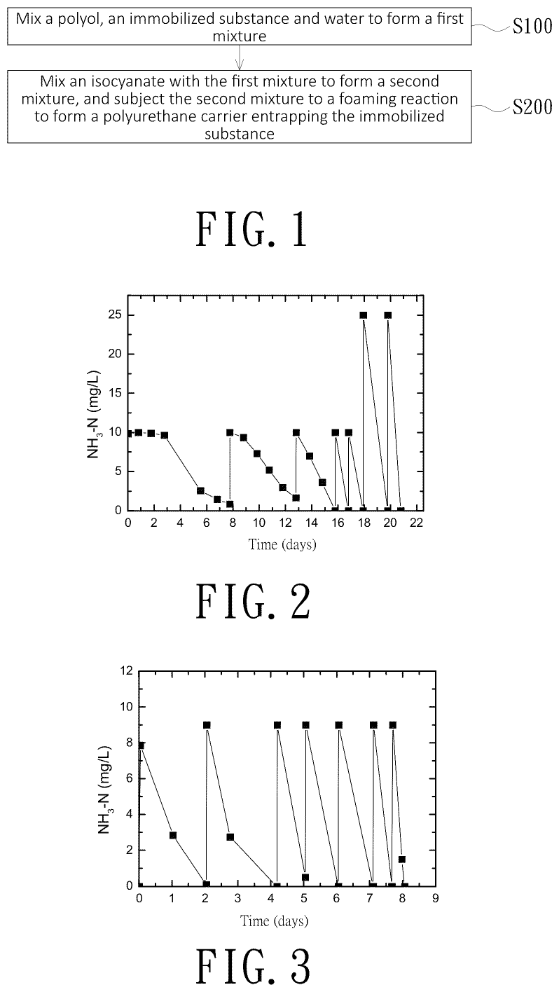

FIG. 1 is a schematic flow diagram of a method for making a polyurethane carrier composite immobilized substance according to an embodiment of the present invention. Referring to FIG. 1, an embodiment of the present invention provides a method for making a polyurethane carrier composite immobilized substance, including: mixing a polyol, an immobilized substance and water to form a first mixture (this is step S100); and mixing an isocyanate with the first mixture to form a second mixture, and subjecting the second mixture to a foaming reaction to form a polyurethane carrier entrapping the immobilized substance (step S200), where a weight of the water accounts for 20% to 50% of a total weight of the polyol and the water, a weight ratio of the isocyanate to a sum of the polyol and the water is between 1:1 and 2:1, a weight ratio of the isocyanate to the polyol is between 1:2 and 4:1, and a weight ratio of the isocyanate to the water is between 1:2 and 6:1. Herein, the second mixture during the foaming reaction is referred to as a foam, and the product formed after the foaming reaction is a polyurethane carrier, that is, a polyurethane foam. In an embodiment of the present invention, the weight ratio between the isocyanate and the polyol can be adjusted as appropriate. For example, when the immobilized substance is an enzyme, the amount of the isocyanate can be reduced.

Specifically, in an embodiment of the present invention, the method for making the polyurethane carrier composite immobilized substance is, for example, two-component PU foaming. Specifically, two liquid-phase materials are mixed, and at the same time, the immobilized substance is uniformly dispersed in the carrier formed by the PU foaming. The above first liquid-phase material is the first mixture formed by the polyol (also referred to as component B), the immobilized substance and the water, and the above second liquid-phase material is the isocyanate (also referred to as component A). The above two liquid-phase materials are mixed to form the second mixture.

It is worth mentioning that in an embodiment of the present invention, the method for making the polyurethane carrier composite immobilized substance can maintain the foaming temperature during the foaming reaction at a low temperature, about below 70° C., for example, between room temperature (25° C.) and 60° C., thereby preventing the efficacy reduction or loss of the immobilized substance due to the high temperature. The temperature during the conventional polyurethane foaming reaction (or it can be understood as the temperature of the foam) is as high as 120° C. to 200° C. At such high temperatures, the efficacy of the immobilized substance will be reduced or lost. In an embodiment of the present invention, the method for making the polyurethane carrier composite immobilized substance sets the weight of the water to account for 20% to 50% of the total weight of the polyol and the water, and sets the weight ratio of the isocyanate to the sum of the polyol and the water to be between 1:2 and 2:1, the weight ratio of the isocyanate to the polyol to be between 1:2 and 4:1, and the weight ratio of the isocyanate to the water to be between 1:2 and 6:1. Since the method for making the polyurethane carrier composite immobilized substance in an embodiment of the present invention uses a relatively high content of water, which can maintain the temperature of the foaming reaction at a low temperature (below 70° C., preferably below 60° C.), this is conducive to maintaining the good activity of the immobilized substance entrapped in the polyurethane carrier.

In other words, the method for making the polyurethane carrier composite immobilized substance in an embodiment of the present invention can make the polyurethane carrier entrapping the immobilized substance. Since the polyurethane carrier has a porous structure with many pores and a large surface area, when a fluid (such as a water body) passes through the polyurethane carrier, the pores facilitate the contact between the fluid and the immobilized substance entrapped in the polyurethane carrier. It is to be noted that although the polyurethane carrier has many pores, the immobilized substance can still be regarded as being entrapped in the polyurethane carrier at a microscopic scale (for example, the surface inside the pores of the polyurethane carrier).

In an embodiment of the present invention, the immobilized substance includes, for example, at least one of a microbial cell, an alga, an enzyme, activated carbon, a catalyst and sludge. In an embodiment of the present invention, the microbial cell may include an aerobic bacterium, a facultative bacterium, a nitrifying bacterium or a denitrifying bacterium, but the present invention is not specifically limited thereto. In an embodiment of the present invention, the enzyme may include catalase, but the present invention is not specifically limited thereto. In an embodiment of the present invention, the catalyst may include at least one of silver, ruthenium (chemical symbol is Ru), manganese, iridium (chemical symbol is Ir) and platinum, but the present invention is not limited thereto. In an embodiment of the present invention, the immobilized substance may include activated carbon and a catalyst, where the activated carbon, for example, acts as a carrier for supporting the catalyst (or called a catalyst support).

As described above, since the method for making the polyurethane carrier composite immobilized substance in an embodiment of the present invention can maintain the temperature at the time of foaming reaction at a low temperature, it is more suitable for entrapping an immobilized substance such as a microbial cell, an enzyme and sludge, and it enables the immobilized substance to be entrapped in the polyurethane carrier while maintaining good activity.

In an embodiment of the present invention, the polyol includes, for example, a polyether polyol, such as polypropylene glycol (PPG) and/or polytetramethylene ether glycol (PTMEG). In an embodiment of the present invention, the polyol has an OH value, for example, between 80 and 300, preferably between 210 and 270.

In an embodiment of the present invention, the isocyanate includes, for example, at least one of methylenediphenyl diisocyanate and a prepolymer of methylenediphenyl diisocyanate. In an embodiment of the present invention, the isocyanate has an NCO value, for example, between 20 and 32, preferably from 24 to 28. In an embodiment of the present invention, the isocyanate is, for example, a modified isocyanate, but the present invention is not specifically limited thereto.

In an embodiment of the present invention, the first mixture further includes, for example, an auxiliary agent, and the above auxiliary agent includes a catalyst and/or a foam stabilizer. In an embodiment of the present invention, the catalyst is, for example, a tertiary amine catalyst or a catalyst containing an organometallic compound (Sn, K or Bi catalyst), and, for example, at least one of dimethylamino cyclohexane, dibutyltin dilaurate and bismuth octoate. A composite catalyst may also be used. In an embodiment of the present invention, the catalyst facilitates the foaming reaction and can thus be used to adjust the speed of the foaming reaction. In an embodiment of the present invention, the foam stabilizer (also referred to as a surfactant) includes, for example, a silicone surfactant or an organic synthetic surfactant. In an embodiment, the organic synthetic surfactant is, for example, Dow Chemical DC-193, DC-198; and Evonik Tegostab B-8950, Tegostab B-8960. In an embodiment of the present invention, the foam stabilizer can adjust the foaming ratio and the pore size of the polyurethane carrier. In an embodiment, addition of the foam stabilizer in a dosage of 2% of the weight of the second mixture can improve the degree of foaming, and the resulting polyurethane carrier has a lower specific gravity and is relatively soft. Increasing the dosage of the foam stabilizer to 10% can make the pores (i.e., bubbles) in the polyurethane carrier smaller and more evenly distributed, and can also increase the compressive stress of the polyurethane carrier. In an embodiment of the present invention, the foaming degree of the polyurethane carrier is, for example, 5 to 10 times, and can reach up to 20 times (2 kg/m³), preferably 6 to 8 times.

In an embodiment of the present invention, the weight of the catalyst accounts for 100 ppm to 500 ppm of the weight of the second mixture, which can be adjusted according to requirements. In an embodiment of the present invention, the weight of the foam stabilizer accounts for 0.1% to 10% of the weight of the second mixture, preferably 0.5% to 5%.

In an embodiment of the present invention, after the step of mixing the isocyanate with the first mixture to form the second mixture, the method for making the polyurethane carrier composite immobilized substance further includes, for example: pouring the second mixture into a mold. Specifically, in an embodiment of the present invention, after the isocyanate and the first mixture are vigorously stirred and mixed to form the second mixture, the second mixture is poured into the mold immediately or within a certain period of time (for example, within 10 minutes to 20 minutes from the start of the mixing of the isocyanate and the first mixture). The second mixture is, for example, subjected to foaming reaction inside the mold (the second mixture undergoing the foaming reaction is a foam). After the foaming reaction is completed, the polyurethane carrier entrapping the immobilized substance is formed.

In an embodiment of the present invention, the mold can be an open mold or a closed mold. The above open mold, for example, does not have an upper cover, allowing the foam to freely rise and expand. The above closed mold, for example, has an upper cover. The foam is formed into a specific shape by the mold, and an integrally formed object can be formed.

In an embodiment of the present invention, during the foaming reaction, the second mixture (the foam) can be stirred, so that the isocyanate and the first mixture can be more thoroughly mixed, facilitating the foaming reaction. In an embodiment, the stirring can be performed using a stirring blade or a magnetic stir bar, but the present invention is not specifically limited thereto.

In summary, since the polyol, the isocyanate and the water are adopted in the present invention, the polyurethane carrier composite immobilized substance, which is easy to form, has good durability and can avoid expansion or rupture, can be produced. In addition, in the present invention, since the weight of water accounts for 20% to 50% of the total weight of the polyol and the water, and the weight ratio of the isocyanate to the sum of the polyol and the water is set to be between 1:2 and 2:1, the weight ratio of the isocyanate to the polyol is between 1:2 and 4:1, and the weight ratio of the isocyanate to water is between 1:2 and 6:1, the temperature of the foaming reaction of the polyurethane carrier can be maintained below 70° C., which helps the immobilized substance entrapped in the polyurethane carrier to have good activity.

In an embodiment of the present invention, at least one of catalyst and foam stabilizer can be used according to different requirements to adjust the pore size, pore uniformity, the speed of the foaming reaction (i.e., the forming speed of the polyurethane carrier, with the foaming and forming time of about 1-2 hours), and the foaming ratio (for example, 5 to 10 times).

In an embodiment of the present invention, the method for making the polyurethane carrier composite immobilized substance can overcome the drawbacks of traditional biological/enzyme/catalyst carriers, including: (1) in traditional biological nitrification/denitrification (AO) processes and anaerobic fermentation processes, a large amount of nitrogen, carbon dioxide and methane is produced, which will cause the algin or PVA beads to expand into balloons or even rupture. However, the present invention will not cause the expansion or deformation of the carrier; (2) when using the present invention to entrap peroxidase enzyme, during the treatment of hydrogen peroxide-containing wastewater, the polyurethane carrier will not react with hydrogen peroxide, nor will it expand or disintegrate due to the generation of a large amount of oxygen; (3) by using the method for making the polyurethane carrier composite immobilized substance in an embodiment of the present invention to produce the polyurethane carrier, the specific gravity of the polyurethane carrier can be flexibly adjusted. If the specific gravity is maintained less than 1, it can be naturally separated from the activated sludge with a specific gravity greater than 1 that will precipitate; (4) traditional PU foams are directly cut from commercially available PU foams and cannot be customized into shapes that meet the requirements. The method for making the polyurethane carrier composite immobilized substance in an embodiment of the present invention can make the polyurethane carrier foam and form in a mold with a designed shape, making it more suitable for application in the purification treatment of aquariums or natural water bodies; (5) semiconductor factories often use activated carbon to treat hydrogen peroxide-containing wastewater. However, because activated carbon is easily damaged by hydrogen peroxide, it can only be used for wastewater with a low concentration of hydrogen peroxide. If activated carbon powder is entrapped in the polyurethane carrier produced in an embodiment of the present invention, the structure of the activated carbon can be effectively protected from being damaged; and (6) traditionally, ion-exchange resin is used to collect cesium in low-pollution nuclear wastewater. However, the incineration of ion-exchange resin requires a temperature higher than the boiling point of cesium, so it can only accumulate in large quantities in nuclear power plants. In recent years, scientists have attempted to use fiber derivatives containing sodium polyacrylate to adsorb cesium, but they are troubled by stack compaction. The above problems can be solved by using the polyurethane carrier produced according to an embodiment of the present invention.

The polyurethane carrier entrapping the immobilized substance produced by the method provided by the present invention for making a polyurethane carrier composite immobilized substance can be used in different applications, including: (1) entrapping known aquarium microbial flora for water quality purification; (2) denitrification of wastewater containing nitrate nitrogen (NO₃—N); (3) nitrification of wastewater containing ammonia nitrogen (NH₃—N); (4) anaerobic and aerobic treatment of COD-containing wastewater; (5) application of entrapping enzymes; (6) application of entrapping catalysts; (7) entrapping sodium polyacrylate derivatives for nuclear wastewater treatment; and (8) polyurethane carriers with customized shapes for purification of natural water bodies.

In an embodiment of the present invention, homogeneous mixing of the second mixture can be achieved using all common batch-used mixing tank, continuous stirred-tank reactor (simply referred to as CSTR) and plug flow reactor. In an embodiment of the present invention, the polyurethane carrier can be used with activated sludge.

### Embodiment 1: Nitrification Treatment of Aquarium Water

In an embodiment of the present invention, the water body source was: a household or corporate aquarium for water quality purification. In this field, the ammonia nitrogen concentration in the water body is generally less than 1-5 mg/L, the BOD value is less than 3 mg/L or negligible, and the nitrate nitrogen in the water body can be directly absorbed by plants for growth without treatment. In an embodiment of the present invention, the microbial sources used in the polyurethane carrier were, for example: (1) a commercially available Microbe® microbial agent, and (2) a pure strain professionally provided by Yusbio Company. In an experiment of this embodiment, for example, a 1-L serum bottle or a 12-L fish tank was adopted for fed-batch operation treatment. The change in the ammonia nitrogen (NH₃—N) concentration in the treated water body is shown in FIG. 2 to FIG. 6.

In an embodiment of the present invention, the first mixture included, for example, 15 g of polypropylene glycol (PPG) with a water content of 35% (OH value of 270) and 2 g of Microbe® microbial agent with a water content of 99%, and, for example, 30 g of methylenediphenyl diisocyanate with a water content of 0% (NCO value of 32) was used. In this embodiment, the foaming ratio of the polyurethane carrier was, for example, 10 times or more (the bubbles were relatively loose, and the texture was relatively hard). In an embodiment of the present invention, the first mixture included, for example, 15 g of polypropylene glycol (PPG) with a water content of 35% (OH value of 270) and 2 g of pure strain provided by Yusbio Company with a water content of 99%, and, for example, 30 g of methylenediphenyl diisocyanate with a water content of 0% (NCO value of 32) was used. In this embodiment, the foaming ratio of the polyurethane carrier was, for example, 10 times or more (the bubbles were relatively loose, and the texture was relatively hard). The present invention is not limited thereto. In another embodiment of the present invention, 100 μg of dimethylamino cyclohexane and 0.2 g of silicone surfactant could be additionally added to the formulation of the above embodiment to enable the foaming reaction to have an appropriate speed and foaming ratio, and to enable the polyurethane carrier to have an appropriate pore size, but the present invention is not specifically limited thereto.

In this embodiment, in the experiment of making the polyurethane carrier using the Microbe® microbial agent (see FIG. 2), the ammonia nitrogen concentration could be treated to zero in each batch. The first batch took 8 days, the time was gradually shortened to 5 days and 3 days for the subsequent batches, and finally, the treatment could be completed in 1 day. When the initial ammonia nitrogen concentration was doubled to 25 mg/L, the reaction time was extended to 2 days, but in the subsequent batches, the time was shortened to 1 day again, indicating that the microorganisms had readapted. FIG. 3 shows the results of moving the polyurethane carrier in FIG. 2 to a 12-L fish tank. The time for complete removal of the ammonia nitrogen concentration was rapidly shortened from 2 days to 1 day, and finally, 10 mg/L ammonia nitrogen could be reduced to zero within about 8 hours. This embodiment could demonstrate that using the Microbe® microbial agent as the microbial source of the polyurethane carrier had an effective nitrification ability.

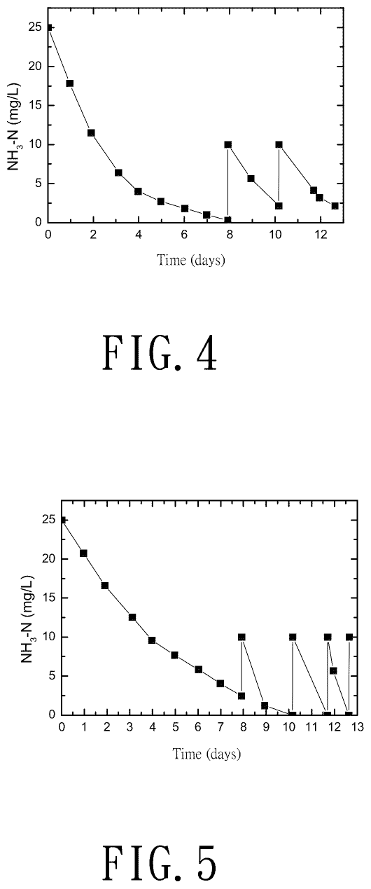

FIG. 4 and FIG. 5 illustrated that the polyurethane carrier containing the Microbe® microbial agent, prepared for the second time in an embodiment of the present invention, also had a nitrification ability. The initial startup time was similar to that in FIG. 2, taking 8 days. Subsequently, it only took 1-2 days per batch to treat 10 mg/L of ammonia nitrogen to zero. It was to be noted that regarding the volume of the polyurethane carrier in an embodiment of the present invention, since the volume of the polyurethane carrier used in FIG. 5 (600 mL) was twice the volume of the polyurethane carrier used in FIG. 4 (300 mL), the nitrification time was shortened to only 1 day.

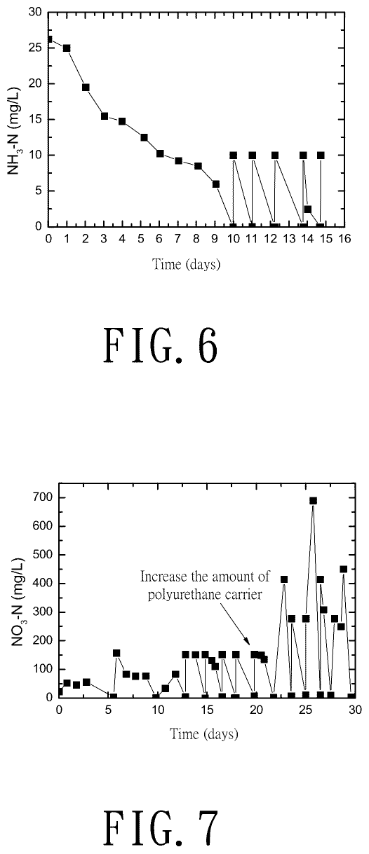

In an embodiment of the present invention, in the experiment of making the polyurethane carrier using the pure strain provided by Yusbio Company (see FIG. 6), the ammonia nitrogen concentration of each batch could be reduced to zero. Initially, it, for example, took about 10 days to complete the treatment. The time required for subsequent batches was gradually shortened to 2 days and 1 day, and finally, the reaction time was reduced to only 6 hours. In an embodiment of the present invention, when the Yusbio strain adapted to an ammonia nitrogen concentration of 25 mg/L, it required longer reaction time initially. However, in later batches, the polyurethane carrier made from it, for example, gradually outperformed the polyurethane carrier made from the commercially available Microbe® microbial agent, demonstrating a more excellent nitrification ability.

### Embodiment 2: Denitrification Experiment of Industrial Wastewater

The water body used in this embodiment was wastewater from a screw factory in southern Taiwan, with a COD value less than 1500 mg/L, an ammonia nitrogen concentration of 50 mg/L, a nitrate nitrogen concentration of 415 mg/L and a conductivity of about 22 mS. In an embodiment of the present invention, the first mixture included, for example, 25 g of polypropylene glycol (PPG) with a water content of 35% (OH value of 270) and 2 g of pure denitrifying strain provided by Azoo Company with a water content of 99%, and, for example, 50 g of methylenediphenyl diisocyanate with a water content of 0% (NCO value of 32) was used. In this embodiment, the foaming ratio of the polyurethane carrier was, for example, 10 times or more (the bubbles were relatively loose, and the texture was relatively hard), and the volume of the produced polyurethane carrier was, for example, 600 ml. The present invention is not limited thereto. In another embodiment of the present invention, 100 μg of dimethylamino cyclohexane and 0.4 g of silicone surfactant could be additionally added to the formulation of the above embodiment to enable the foaming reaction to have an appropriate speed and foaming ratio, and to enable the polyurethane carrier to have an appropriate pore size, but the present invention is not specifically limited thereto.

In an experiment of this embodiment, for example, 200 mL of the above 600 mL polyurethane carrier was used for the experiment, and, for example, a 1-L serum bottle was adopted for fed-batch operation. It could be seen from FIG. 7 that the initial use of nitrate nitrogen at a lower concentration (152 mg/L) for acclimation took 6 days at first, then the time was gradually shortened to 5 days, 3 days and 2 days, and finally, the treatment was stably completed in 1 day. After the acclimation was completed, for example, the wastewater from the screw factory was directly used, with the nitrate nitrogen concentration raised to 415 mg/L and the C/N ratio increased from 4 to 10. The results showed that the reaction time was further shortened.

The above results are organized as shown in Table 1. During the acclimation period, the nitrate nitrogen concentration was gradually raised from 152 mg/L to 277 mg/L. With the completion of acclimation, the retention time was significantly shortened. After performing actual wastewater tests and increasing the C/N ratio to 10, the reaction time was halved again, and it only took 0.88 days to achieve the removal of nitrate nitrogen.

TABLE 1
Results of denitrification experiments on simulated wastewater
and actual industrial wastewater

| | | | | Average | Capacity |
|---|---|---|---|---|---|
| | | Retention | | retention | (kg |
| | NO₃- | time | C/N | time | N/m³- |
| | N(mg/L) | (days) | ratio | (days) | PU-day) |
| Simulated | 152 | 1.99 | 4 | 1.78 | 0.085 |
| wastewater | 152 | 1.71 | 4 | | |
| KNO₃, 1.1 | 152 | 1.38 | 4 | | |
| g/L | 152 | 1.87 | 4 | | |
| | 152 | 1.94 | 4 | | |
| Simulated | 277 | 1.43 | 4 | 1.53 | 0.181 |
| wastewater | 277 | 1.47 | 4 | | |
| KNO₃, 2.0 | 277 | 1.69 | 4 | | |
| g/L | | | | | |
| Actual | 415 | 0.74 | 10 | | |
| industrial | 415 | 1.02 | 10 | 0.88 | 0.471 |
| wastewater | | | | | |
| (screw | | | | | |
| factory) | | | | | |

In this embodiment, the water source for the experiment was, for example, simulated wastewater containing nitric acid, but it was different from the above experiment in that, for example, a higher amount of entrapped sludge was used in one experiment of this embodiment, and a large number of bubbles could be observed during cultivation. The sludge source in this embodiment was, for example, centrifugal sludge of a petrochemical plant. The proportions of the sludge and polyol were comparable. Specifically, the first mixture included, for example, 40 g of polypropylene glycol (PPG) with a water content of 35% (OH value of 210) and 10 g of the above sludge with a water content of 80%, and, for example, 50 g of methylenediphenyl diisocyanate with a water content of 0% (NCO value of 25) was used. In this embodiment, the foaming ratio of the polyurethane carrier was, for example, 7 times (the bubbles were moderately distributed, and the texture was relatively soft). The present invention is not limited thereto. In another embodiment of the present invention, 100 μg of dimethylamino cyclohexane and 0.5 g of silicone surfactant could be additionally added to the formulation of the above embodiment. In another embodiment, the first mixture included, for example, 42 g of polypropylene glycol (PPG) with a water content of 35% (OH value of 210) and 30 g of the above sludge with a water content of 80%, and, for example, 30 g of methylenediphenyl diisocyanate with a water content of 0% (NCO value of 25) was used. In this embodiment, the foaming ratio of the polyurethane carrier was, for example, 3 times (the bubbles were relatively compact, and the texture was relatively soft). The present invention is not limited thereto. In another embodiment of the present invention, 100 μg of dimethylamino cyclohexane and 0.4 g of silicone surfactant could be additionally added to the formulation of the above embodiment. In the experiment of this embodiment, for example, a fed-batch experiment was also performed using a 1-L serum bottle. The initial NO₃—N concentration was 250 mg/L, and after 16.5 hours, the NO₃—N concentration was decreased to 60 mg/L. The volume loading of this reactor is 0.3 kg N/m³·d, and the filling rate is 40%.

### Embodiment 3: Nitrification Experiment Using a PU Foam Carrier Made of Mixed Bacteria

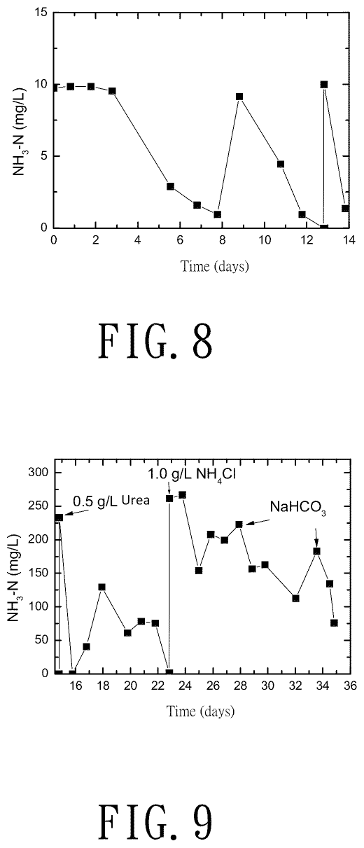

In this embodiment, the water body source for the experiment was simulated wastewater containing ammonia nitrogen, and tests were performed in two scenarios of high and low concentrations. Similar to the above embodiments, in the embodiments shown in FIG. 8 and FIG. 9, a fed-batch experiment was also performed using a 1-L serum bottle and, for example, 200 mL of a polyurethane carrier containing an immobilized substance of mixed bacteria (using activated sludge from the wastewater of a certain petrochemical plant) was filled. The low-concentration ammonia nitrogen in the simulated wastewater used in this embodiment was set to, for example, 10 mg/L (see FIG. 8), and the high-concentration ammonia nitrogen was set to, for example, 250 mg/L (see FIG. 9).

In an embodiment of the present invention, the first mixture included, for example, 50 g of polypropylene glycol (PPG) with a water content of 35% (OH value of 210) and 4 g of activated sludge from a petrochemical plant with a water content of 99%, and, for example, 50 g of methylenediphenyl diisocyanate with a water content of 0% (NCO value of 25) was used. In this embodiment, the foaming ratio of the polyurethane carrier was, for example, 7 times (the bubbles were moderately distributed, and the texture was relatively soft). The present invention is not limited thereto. In another embodiment of the present invention, 100 μg of dimethylamino cyclohexane and 0.5 g of silicone surfactant could be additionally added to the formulation of the above embodiment.

It could be seen from FIG. 8 that although the amount of the polyurethane carrier filled in this batch experiment was relatively small, resulting in a slow initial startup speed, similar to the previous experiments, it took about 8 days for the complete degradation of ammonia nitrogen. After the acclimation was completed, the treatment speed gradually increased to 4 days and 1 day, entering a stable state. In the embodiment shown in FIG. 9, for example, 0.5 g/L of urea was first added as a nitrogen source. After it was completely degraded in about 8 days, 1.0 g/L of ammonium chloride was then added as a nitrogen source, and during this process, adding baking soda (NaHCO₃) could facilitate the degradation reaction. Therefore, it could be seen from the embodiment shown in FIG. 9 that under high-concentration ammonia nitrogen conditions, the reaction time was extended to 8 days, and the effect of reducing the ammonia nitrogen concentration was less significant. The effect of reducing the ammonia nitrogen concentration could be enhanced by adding baking soda.

### Embodiment 4: Anaerobic Experiment Using a Polyurethane Carrier Made of Mixed Bacteria

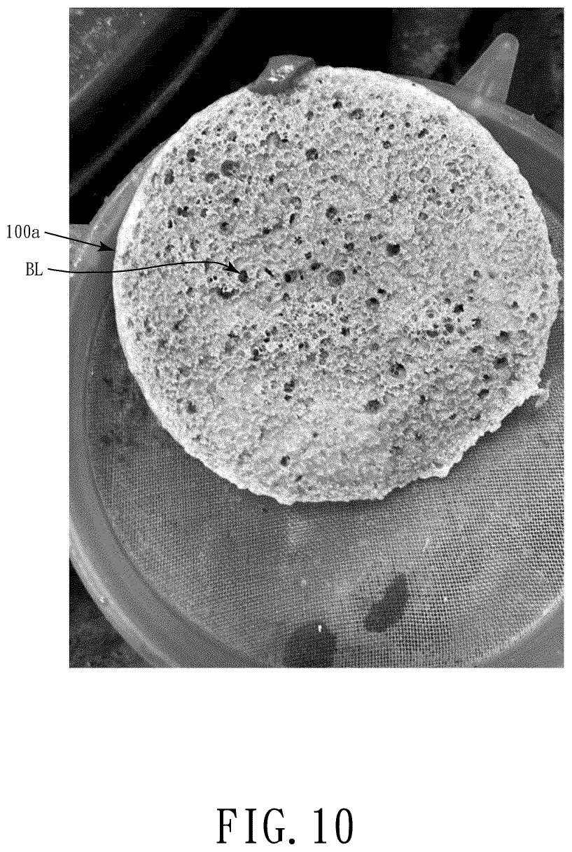

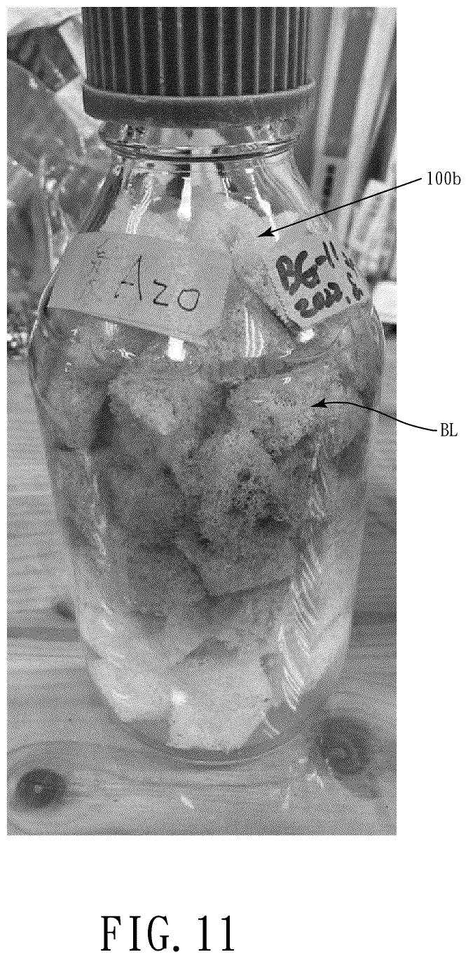

The bacterial source used in this embodiment is precipitated sludge in the upflow anaerobic sludge bed (UASB) tank of a certain petrochemical plant in Miaoli. The methane recovery rate of the anaerobic reaction in this embodiment reached 46%, which could demonstrate that the microbial cells underwent the anaerobic reaction. In an embodiment of the present invention, the first mixture included, for example, 30 g of polypropylene glycol (PPG) with a water content of 35% (OH value of 210) and 2 g of activated sludge from a petrochemical plant with a water content of 80%, and, for example, 30 g of methylenediphenyl diisocyanate with a water content of 0% (NCO value of 25) was used. In this embodiment, the foaming ratio of the polyurethane carrier was, for example, 7 times (the bubbles were moderately distributed, and the texture was relatively soft). The present invention is not limited thereto. In another embodiment of the present invention, 100 μg of dimethylamino cyclohexane and 0.3 g of silicone surfactant could be additionally added to the formulation of the above embodiment. In an embodiment of the present invention, the first mixture included, for example, 50 g of polypropylene glycol (PPG) with a water content of 35% (OH value of 210) and 20 g of activated sludge from a petrochemical plant with a water content of 80%, and, for example, 50 g of methylenediphenyl diisocyanate with a water content of 0% (NCO value of 25) was used. In this embodiment, the foaming ratio of the polyurethane carrier was, for example, 4 times (the bubbles were relatively compact, and the texture was relatively soft). The present invention is not limited thereto. In another embodiment of the present invention, 100 μg of dimethylamino cyclohexane and 0.5 g of silicone surfactant could be additionally added to the formulation of the above embodiment. FIG. 10 shows a state of a polyurethane carrier 100a after being soaked in sludge according to an embodiment of the present invention. It could be seen from FIG. 10 that the pores of the polyurethane carrier 100a appeared black BL, indicating that anaerobic bacteria had grown therein. In an embodiment of the present invention, when the polyurethane carrier 100a was wrung by hand, a large amount of sludge was found to flow out. Even after being wrung and washed by hand more than 5 times, there was still residual sludge in the pores of the polyurethane carrier 100a. FIG. 11 shows that the same bacterial source as in FIG. 10 was used for denitrification purposes and appeared black BL after standing for a long time, indicating that it was in an anaerobic state. The reason why the black BL appeared as above was presumably due to the formation of black sulfide in an anaerobic state. The focus of this embodiment is to emphasize that conventional carriers are prone to expansion and deformation during use, while the polyurethane carrier 100a in an embodiment of the present invention still maintains a solid structure after use, without obvious deformation or expansion, thus maintaining its structural stability.

### Embodiment 5: Treatment of Hydrogen Peroxide-Containing Wastewater Using a Polyurethane Carrier Entrapping a Silver Ruthenium Catalyst

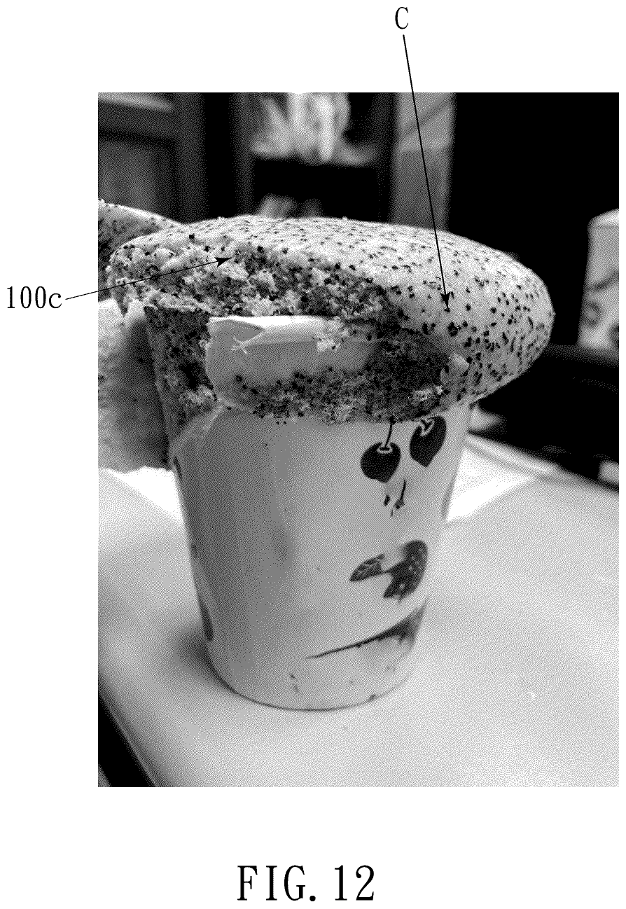

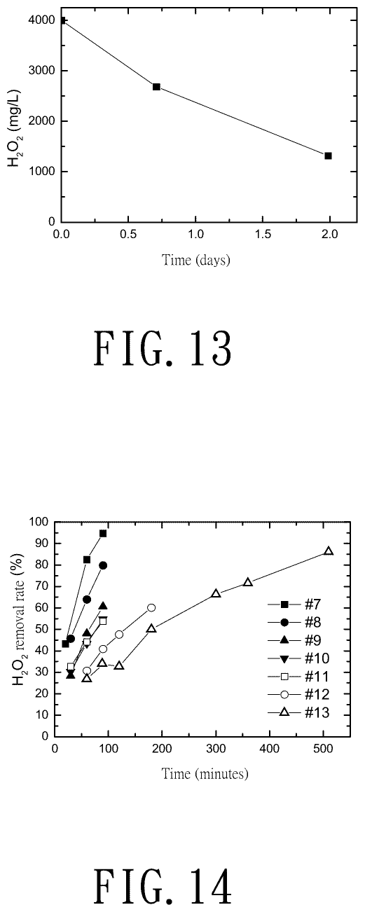

The water body source used in this embodiment was simulated wastewater from a semiconductor factory, i.e., the wastewater generated from cleaning wafers with hydrogen peroxide (H₂O₂). The H₂O₂ concentration in the wastewater in this embodiment was, for example, 4,000 mg/L, and the immobilized substance was, for example, catalyst C, where the amount of catalyst C used was, for example, 4.14 g, for example, to synthesize 400 mL of the polyurethane carrier 100c (FIG. 12). In an embodiment of the present invention, the first mixture included, for example, 10 g of polypropylene glycol (PPG) with a water content of 35% (OH value of 270), and, for example, 10 g of methylenediphenyl diisocyanate with a water content of 0% (NCO value of 32) and 4.14 g of catalyst C were used (in this embodiment, catalyst C was mixed with methylenediphenyl diisocyanate, for example, and then mixed with the first mixture). In this embodiment, the foaming ratio of the polyurethane carrier 100c was, for example, more than 7 times (the bubbles were moderately distributed, and the texture was relatively hard). The present invention is not limited thereto. In another embodiment of the present invention, 100 μg of dimethylamino cyclohexane and 0.1 g of silicone surfactant could be additionally added to the formulation of the above embodiment. In the embodiment shown in FIG. 12, the polyurethane carrier 100c was foamed and formed, for example, in a paper cup, but the present invention is not specifically limited thereto. In an embodiment of the present invention, the preparation process of catalyst C included, for example, first implanting silver and manganese into activated carbon, and then uniformly coating silver and ruthenium in the pores of the activated carbon through a surfactant, where the concentration of ruthenium was, for example, 3 ppm. In an embodiment of the present invention, the result of treating hydrogen peroxide-containing wastewater using the polyurethane carrier 100c containing this catalyst C is shown in FIG. 13. It could be seen from FIG. 13 that since the total amount of the ruthenium catalyst was only 12.42×10⁻⁶ g, it took many days of reaction to achieve a significant removal effect. According to the experimental results, the table below shows that each gram of ruthenium could treat nearly 75 kilograms of hydrogen peroxide per day at most. It was to be noted that the catalyst C used in an embodiment of the present invention was, for example, a supported catalyst with a ruthenium catalyst supported on activated carbon, but the present invention is not specifically limited thereto. In another embodiment of the present invention, the catalyst C may not be supported on a carrier.

TABLE 2
Experimental results of the polyurethane carrier including the
catalyst

| | | | H₂O₂ | Capacity (kg |
|---|---|---|---|---|
| Duration | Titrated | H₂O₂ | removed | H₂O₂/g |
| (days) | KMO₄ (mL) | (mg/L) | (g) | Ru-day) |
| 0 | 7.9 | 4000 | | |
| 0.71 | 5.3 | 2684 | 0.658 | 74.82 |
| 1.99 | 2.6 | 1316 | 0.684 | 43.07 |

### Embodiment 6: Treatment of Hydrogen Peroxide-Containing Wastewater Using a Catalase Enzyme

In an embodiment of the present invention, for example, simulated wastewater was adopted, which simulated the wastewater generated after semiconductor factories clean wafers with hydrogen peroxide. In an embodiment of the present invention, the volume of the simulated wastewater was, for example, 500 mL, and the H₂O₂ concentration reached 4,000 mg/L. In an embodiment of the present invention, during the preparation process of the foaming reaction of the polyurethane carrier, the first mixture included, for example, 30 g of polypropylene glycol (PPG) with a water content of 35% (OH value of 270) and 7 g of catalase enzyme with a water content of 99.8%, and, for example, 25 g of methylenediphenyl diisocyanate with a water content of 0% (NCO value of 32) was used to produce 1 L of polyurethane (PU) carrier, where 200 mL of the 1-L polyurethane carrier produced by the above method was used as the filling filter material of the filter. In this embodiment, the foaming ratio of the polyurethane carrier was, for example, 4 times (the bubbles were relatively compact, and the texture was relatively hard). The present invention is not limited thereto. In another embodiment of the present invention, 100 μg of dimethylamino cyclohexane and 0.3 g of silicone surfactant could be additionally added to the formulation of the above embodiment. In an embodiment of the present invention, the outlet pipe was directly inserted into the polyurethane carrier through, for example, an aquarium water pump to treat the wastewater.

As shown in the results of Table 3 and Table 4 below, the polyurethane carrier in an embodiment of the present invention exhibited excellent efficacy in treating hydrogen peroxide-containing wastewater. Its initial treatment capacity reached 0.152 kg H₂O₂/g·day (as shown in Table 3), which was significantly higher than the treatment capacity of subsequent reactions (0.014-0.026 kg H₂O₂/g·day) (as shown in Table 4), approximately 7.5 times higher. This difference in efficacy might be due to the fact that the enzyme that had not fully reacted with the polyol remained inside the pores of the polyurethane carrier or unreacted enzyme remained deep within the polyurethane carrier, which directly provided the decomposition effect of additional subsequent reactions without mass transfer resistance.

TABLE 3
Experimental results of efficacy testing of the polyurethane
carrier including catalase, Experiment 1

| | | | H₂O₂ | Capacity (kg |
|---|---|---|---|---|
| Duration | Titration | H₂O₂ | removed | H₂O₂/g-E- |
| (minutes) | KMO₄ (mL) | (mg/L) | (g) | day) |
| 0 | 9.5 | 4000 | 2.000 | |
| 10 | 2.5 | 1053 | 1.474 | 0.152 |
| 20 | 1.5 | 632 | 0.211 | 0.022 |

Note:
E in Table 3 refers to the enzyme.

TABLE 4
Experimental results of efficacy testing of the polyurethane carrier
including catalase, Experiment 2

| | | | H₂O₂ | Capacity (kg |
|---|---|---|---|---|
| Duration | Titration | H₂O₂ | removed | H₂O₂/g-E- |
| (minutes) | KMO₄ (mL) | (mg/L) | (g) | day) |
| 0 | 9.5 | 4000 | 2.000 | |
| 10 | 8.3 | 3495 | 0.253 | 0.026 |
| 60 | 5 | 2105 | 0.695 | 0.014 |

Note:
E in Table 4 refers to the enzyme.

### Embodiment 7: Treatment of High-Concentration Hydrogen Peroxide-Containing Wastewater Using a Catalase Enzyme

In an embodiment of the invention, simulated wastewater was adopted, which simulated high-concentration hydrogen peroxide-containing wastewater generated in the advanced semiconductor encapsulating process. In an embodiment of the invention, the volume of the simulated wastewater was, for example, 500 mL, and the hydrogen peroxide concentration was, for example, 35,000 mg/L. In an embodiment of the present invention, during the preparation process of the foaming reaction of the polyurethane carrier, the first mixture included, for example, 10 g of polypropylene glycol (PPG) with a water content of 35% (OH value of 270) and 4 g of catalase enzyme with a water content of 99.8%, and, for example, 8 g of methylenediphenyl diisocyanate with a water content of 0% (NCO value of 32) was used to produce 250 mL of polyurethane (PU) carrier (the foaming ratio was, for example, 4 times, the bubbles were relatively compact, and the texture was relatively hard), and 200 mL of the 250 mL polyurethane carrier produced by the above was used as the filling filter material of the filter. The present invention is not limited thereto. In another embodiment of the present invention, 100 μg of dimethylamino cyclohexane and 0.1 g of silicone surfactant could be additionally added to the formulation of the above embodiment. In an embodiment of the present invention, during the experiment, for example, a standard aquarium filter and a 5-L fish tank were used as reactors to provide a more uniform water flow circulation, but the present invention is not specifically limited thereto. In an embodiment of the present invention, in the initial stage of the experiment, for example, hydrogen peroxide with a concentration of 35,000 mg/L was used for circular treatment each time. Since the hydrogen peroxide could be rapidly treated within 20 minutes in the first 6 times, it is not shown in FIG. 14; the tests from the 7th to the 13th time are shown in FIG. 14.

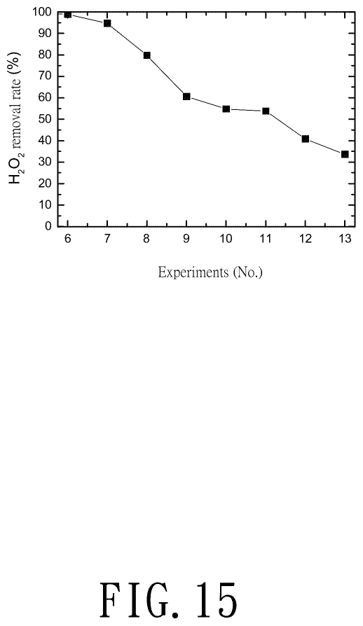

In an embodiment of the present invention, the polyurethane carrier exhibited excellent stability and efficacy in the treatment of hydrogen peroxide-containing wastewater. In an embodiment of the present invention, the initial highest specific activity of the polyurethane carrier reached 8,970 units (μmol H₂O₂/min), and the average specific activity was 1,254 units (see FIG. 15). It was worth mentioning that although the specific activity value of the enzyme entrapped in the polyurethane carrier was only 3.5% of the specific activity (35,000 units to 37,000 units) when the enzyme was directly used, the polyurethane carrier in an embodiment of the present invention could effectively retain the enzyme within the polyurethane carrier, helping to prevent enzyme loss, thereby significantly improving the overall use efficiency of the enzyme, and enabling the capacity (i.e., treatment capacity) to reach 1.022 kg H₂O₂/mL·day, which was only 50%, compared with the conventional free enzyme 2.4 kg/H₂O₂/mL·day. This showed that the polyurethane carrier entrapping the enzyme produced in an embodiment of the present invention could effectively immobilize the free enzyme in the polyurethane carrier and improve the overall use efficiency of the enzyme. In an embodiment, performing the foaming reaction under refrigeration (e.g., below 10° C.) could enhance the effect of the polyurethane carrier entrapping the enzyme.

In 13 consecutive experiments performed in an embodiment of the present invention, the cumulative amount of hydrogen peroxide passing through reached 148.75 g per time×13 times, and 127.88 g/time×13 times were successfully removed, with the treatment efficiency of 86%.

In terms of treatment capacity (capacity), in an embodiment of the present invention, a comparison was made between the polyurethane carrier entrapping the enzyme and the free enzyme (i.e., the enzyme was not entrapped in the polyurethane). In an embodiment of the present invention, based on 300 mL of hydrogen peroxide with a concentration of 9,000 mg/L produced by Company T, within 20-minute retention time, a solution containing 400 μL of the free enzyme was required to completely remove the hydrogen peroxide in the water. After calculation, the treatment capacity of the free enzyme obtained was 6.35 g H₂O₂/mL. However, in an embodiment of the present invention, only a polyurethane carrier containing 4 grams of the enzyme with a water content of 99.8% was needed to completely remove the hydrogen peroxide in the water. After calculation, the treatment capacity of the polyurethane carrier entrapping the enzyme could be obtained, reaching 447.6 g H₂O₂/mL. This showed that the treatment efficiency of the polyurethane carrier entrapping the enzyme in an embodiment of the present invention was 80 times that of directly using the free enzyme, fully demonstrating that the polyurethane carrier significantly improved the utilization efficiency of the enzyme.

### Embodiment 8: Experiment on Delaying Catalase Enzyme Loss Using a Polyurethane Carrier

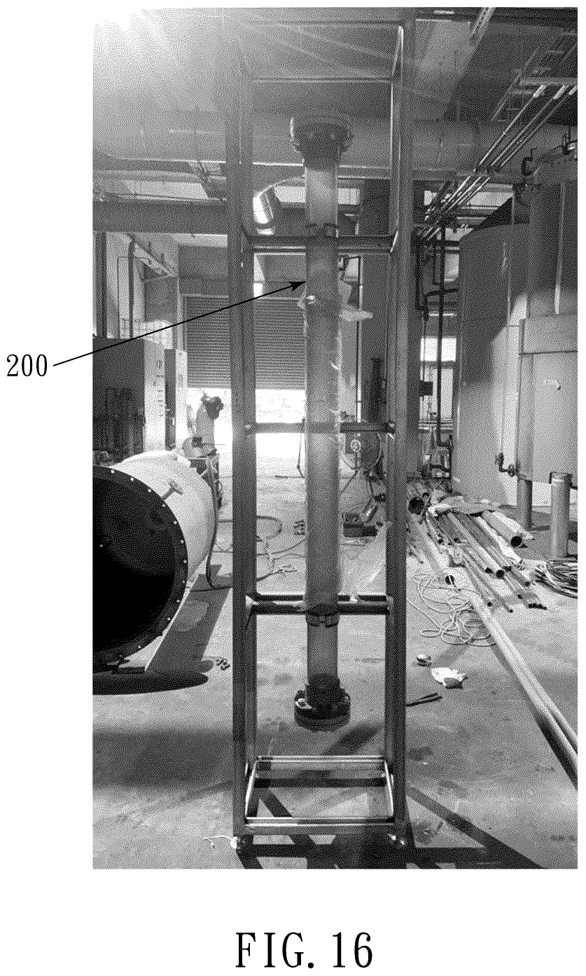

In an embodiment of the present invention, by utilizing the partition principle of the chromatographic column, the enzyme passing through with water could be retained in the polyurethane carrier and the enzyme could be released slowly. In the experiment of an embodiment of the present invention, a column 200 (shown in FIG. 16) with a diameter of 4 inches and a length of 4 meters, made of a material such as PVC, was used. In an embodiment of the present invention, for example, the interior of the column 200 was filled with a wetted polyurethane carrier (not shown in figures), but the polyurethane carrier had no enzyme function (i.e., the enzyme entrapped in the polyurethane carrier had been completely inactivated). In an embodiment of the present invention, the column 200 was supplied with water by an aquarium water pump at a flow rate of about 4,000 LPH. The time required for water to enter one end of the column 200 and flow out of the other end of the column 200 was about 40 seconds. Next, in an embodiment of the present invention, 1 liter of an enzyme-containing solution was injected into one end of the column 200, and samples were taken every five seconds to measure chemical oxygen demand (COD). In an embodiment of the present invention, the peak value of COD was measured approximately one and a half minutes after water entered the column 200. This showed that the polyurethane carrier could retain the enzyme and effectively extend the action time of the enzyme, which could save half the amount of the enzyme used.

### Embodiment 9: Testing of Compatibility Between PVA and a Polyurethane Carrier

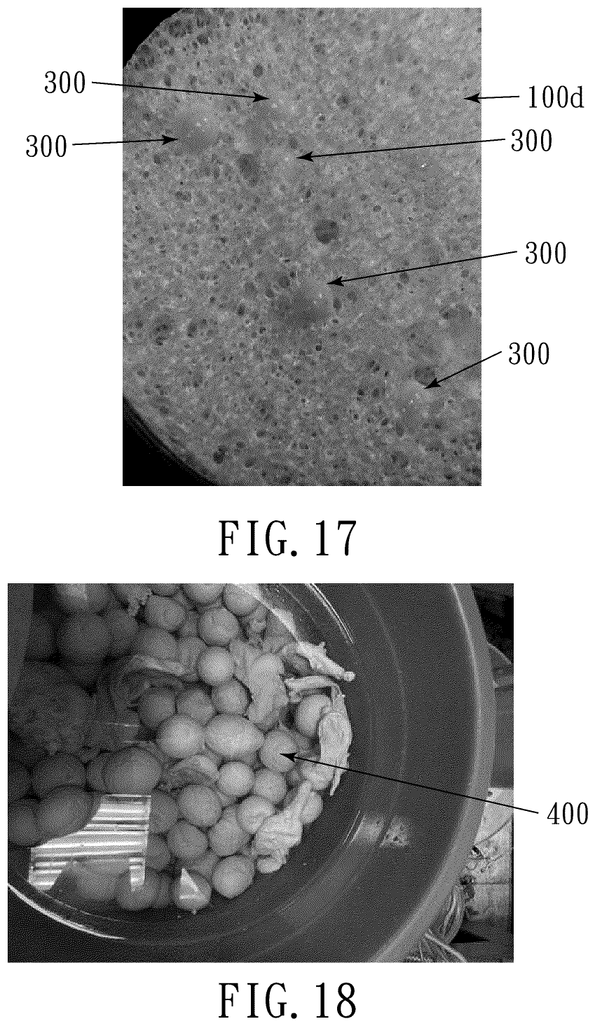

In the experiment of an embodiment of the present invention, PVA beads 300 entrapping microorganisms were successfully entrapped inside a polyurethane carrier 100d, and the number of the PVA beads 300 in this embodiment was, for example, plural. FIG. 17 is a photograph of the polyurethane carrier 100d including (i.e. entrapping) multiple PVA beads 300 according to an embodiment of the present invention. Specifically, in this embodiment, after isocyanate was mixed with a first mixture to form a second mixture, the second mixture was uniformly mixed, for example, using a high-speed mixer, and the multiple PVA beads 300 were poured, for example, during stirring. Then the uniformly mixed PVA beads 300 and the second mixture (or the foam that had started the foaming reaction) were poured into a mold, and the second mixture was subjected to the foaming reaction to form the polyurethane carrier 100d entrapping the multiple PVA beads 300. In an embodiment of the present invention, the PVA beads 300 used, for example, entrapped microorganisms such as bacterial cells, algae, cells, etc., but the present invention is not limited thereto. In an embodiment of the present invention, the PVA beads 300 could be produced by the method described in Republic of China Patent No. 1759755, but the present invention is not specifically limited thereto. Although the PVA beads 300 were entrapped in the polyurethane carrier 100d in the above embodiment, the present invention is not limited thereto.

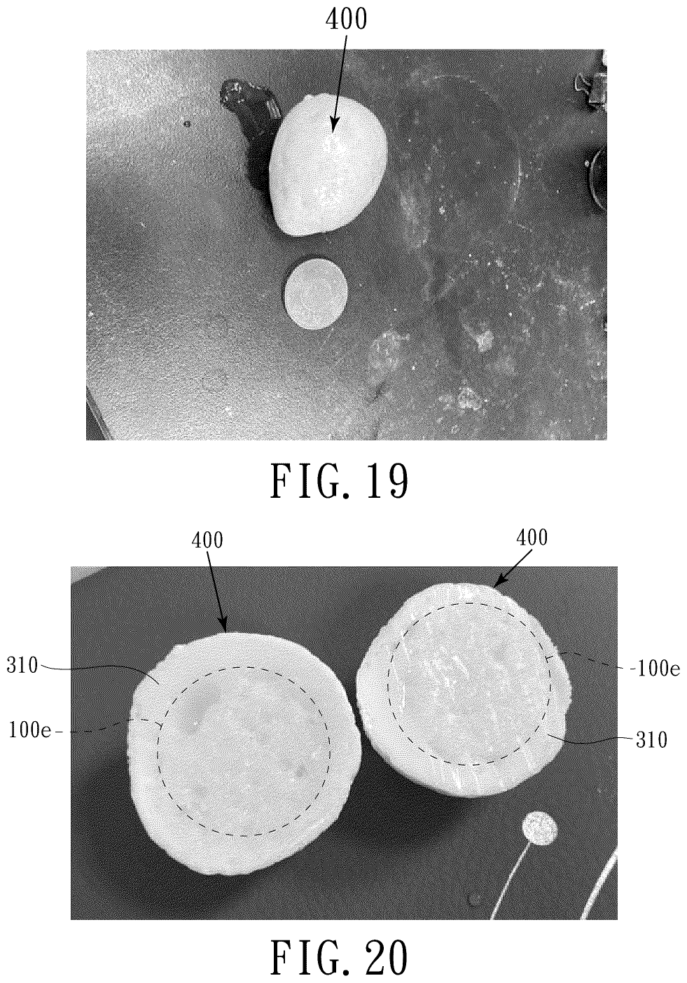

In another embodiment of the present invention, the polyurethane carrier 100e could also be entrapped inside a PVA material 310 to form a spherical object 400, as shown in FIG. 18, FIG. 19 and FIG. 20. FIG. 18 is a photograph of the spherical object 400 immersed in an aqueous solution according to an embodiment of the present invention. The above aqueous solution included, for example, at least one hardening agent, and the hardening agent is, for example, alkali metal ions, alkaline-earth metal ions or a mixture thereof. FIG. 19 is a photograph of the spherical object 400 and a New Taiwan Dollar fifty-dollar coin according to an embodiment of the present invention. As shown in FIG. 19, the spherical object 400 entrapping the polyurethane carrier 100e was about twice the size of a fifty-dollar coin, with a diameter of about 3 centimeters to 5 centimeters. FIG. 20 is a schematic cross-sectional view of the spherical object 400 including the polyurethane carrier 100e inside according to an embodiment of the present invention. In an embodiment of the present invention, for example, the polyurethane carrier 100e was produced first. Subsequently, the polyurethane carrier 100e was mixed with the PVA material 310 and then kneaded into a sphere (not shown in figures). After that, the sphere was, for example, subjected to an immobilization treatment with an aqueous solution containing boric acid, to obtain the spherical object 400. From the cross-sectional view of the spherical object 400 in FIG. 20, it could be seen that the pores of the polyurethane carrier 100e were not very distinct. It was conjectured that this might be due to some of the PVA material 310 entering the above pores when the polyurethane carrier 100e and the PVA material 310 were mixed. It was worth mentioning that the spherical object 400 in this embodiment was suitable for groundwater treatment because it had a relatively large volume and a relatively high mass transfer resistance, which facilitated the action of the immobilized substance (not shown in figures) entrapped in the polyurethane carrier 100 e for a long time.

### Embodiment 10: PU Foamed Carrier for Natural Water Body Purification

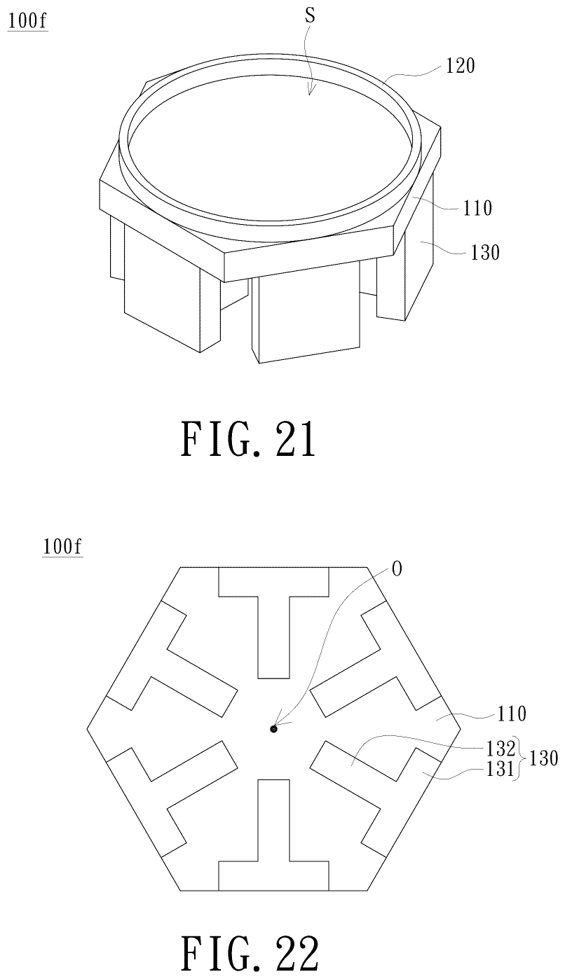

In an embodiment of the present invention, the water body source was a natural water body, and a polyurethane carrier 100f was designed, for example, to simulate a floating island in the water in landscape design. FIG. 21 and FIG. 22 show the integrally formed polyurethane carrier 100f according to an embodiment of the present invention. In an embodiment of the present invention, the interior of the polyurethane carrier 100f could entrap nitrifying bacteria or denitrifying bacteria in the aquarium (not shown in figures). In an embodiment of the present invention, the shape of the polyurethane carrier 100f could be customized according to requirements. For example, on the top of the polyurethane carrier 100f, a space S for placing vegetation (not shown in figures) could be designed according to landscape requirements, as shown in FIG. 21. In an embodiment of the present invention, the polyurethane carrier 100f was, for example, integrally formed and included, for example, a base plate 110, a wall 120, and multiple brackets 130, where the base plate 110 was, for example, a hexagonal plate. In this embodiment, the wall 120 of the polyurethane carrier 100f was disposed, for example, on a side of the base plate 110 away from the brackets 130. The wall 120 could form the space S by enclosing. In this embodiment, the wall 120 was, for example, in a circular annular shape, but the present invention is not specifically limited thereto. In an embodiment, potted plants (not shown in figures) could be placed in the space S formed by the wall 120 and be in contact with the base plate 110. In an embodiment of the present invention, the brackets 130 were, for example, disposed on a side of the base plate 110 away from the wall 120, and were suitable for supporting the potted plants (not shown in figures), the base plate 110 and the wall 120. In this embodiment, the number of the brackets 130 was, for example, 6, and each bracket 130 had, for example, a first sub-bracket 131 and a second sub-bracket 132 connected, where each first sub-bracket 131 was disposed, for example, along an outer contour of the base plate 110, and each second sub-bracket 132 extended, for example, along a direction radiating from a center O of the base plate 110, and intersected, for example, the first sub-bracket 131 perpendicularly, so that the first sub-bracket 131 and the second sub-bracket 132 formed, for example, a T-shaped structure. The present invention does not specifically limit the shape of the polyurethane carrier 100f, including the shape and number of the base plate 110, the wall 120 and the brackets 130, as well as the application form of the polyurethane carrier 100f. In another embodiment of the present invention, a water-spraying device (not shown in figures) could be designed to be disposed in the middle of the polyurethane carrier 100f to agitate the water surface and increase the dissolved oxygen content in the water body, but the present invention is not specifically limited thereto.

In summary, since the polyol, the immobilized substance, the isocyanate and the water are adopted in the present invention, the polyurethane carrier, which is easy to form, has good durability and can avoid expansion or rupture, can be produced. In addition, in the present invention, since the weight of water accounts for 20% to 50% of the total weight of the polyol and the water, and the weight ratio of the isocyanate to the sum of the polyol and the water is set to be between 1:2 and 2:1, the weight ratio of the isocyanate to the polyol is between 1:2 and 4:1, and the weight ratio of the isocyanate to water is between 1:2 and 6:1, the temperature of the foaming reaction of the polyurethane carrier can be maintained below 70° C., which helps the immobilized substance entrapped in the polyurethane carrier to have good activity.

### LITERATURE REVIEW

1. Chen, K.-C. and Lin, Y.-F. 1994. Immobilization of microorganisms or enzymes in polyvinyl alcohol beads. U.S. Pat. No. 5,290,693.
2. Kuraray Co., Ltd. 2012. Shaped article made of porous hydrogel, method for producing the same and use thereof. Republic of China Invention U.S. Pat. No. 1,579,035.
3. Van Dinh, P. and Bach, L. T., Immobilized bacteria by using PVA (Polyvinyl alcohol) crosslinked with Sodium sulfate. The International Journal of Engineering Science, 7 (1): 41-47, 2014.
4. Shenzhen Changlong Technology Co., Ltd. 2017. Reinforcing and toughening polyvinyl alcohol (PVA) spherical microbial carrier and preparation method thereof. Mainland China Published Patent No. CN107937381A.
5. Shandong University. 2019. Method for efficiently treating nitrogen-containing wastewater based on DNRA-Anammox immobilized pellets. Mainland China Patent No. CN110078206B.
6. Hwang, Sz-chwun, Lin, Yun-huin, Chen, Bo-an, et al. 2014. Development of a novel microbial immobilization method using anionic polyurethane. Republic of China Invention Patent No. I425050.

Although the present invention has been disclosed as above by way of embodiments, they are not intended to limit the present invention. A person having ordinary knowledge in the technical field to which the present invention belongs may make some changes and modifications without departing from the spirit and scope of the present invention. Therefore, the protection scope of the present invention shall be determined by the scope of the attached patent claims.

What is claimed is:

1. A method for making a polyurethane carrier composite immobilized substance, comprising:
mixing a polyol, an immobilized substance and water to form a first mixture; and
mixing an isocyanate with the first mixture to form a second mixture, and subjecting the second mixture to a foaming reaction to form a polyurethane carrier entrapping the immobilized substance,
wherein a weight of the water accounts for 20% to 50% of a total weight of the polyol and the water, a weight ratio of the isocyanate to a sum of the polyol and the water is between 1:2 and 2:1, a weight ratio of the isocyanate to the polyol is between 1:2 and 4:1, and a weight ratio of the isocyanate to the water is between 1:2 and 6:1.

2. The method for making a polyurethane carrier composite immobilized substance according to claim 1, wherein after the step of mixing the isocyanate with the first mixture to form the second mixture, the method further comprises: pouring the second mixture into a mold.

3. The method for making a polyurethane carrier composite immobilized substance according to claim 1, wherein the polyol comprises a polyether polyol.

4. The method for making a polyurethane carrier composite immobilized substance according to claim 1, wherein the polyol has an OH value between 80 and 300.

5. The method for making a polyurethane carrier composite immobilized substance according to claim 1, wherein the isocyanate comprises at least one of methylenediphenyl diisocyanate and a prepolymer of methylenediphenyl diisocyanate.

6. The method for making a polyurethane carrier composite immobilized substance according to claim 1, wherein the isocyanate has an NCO value between 20 and 32.

7. The method for making a polyurethane carrier composite immobilized substance according to claim 1, wherein the immobilized substance comprises at least one of a microbial cell, an enzyme, activated carbon, a catalyst and sludge.

8. The method for making a polyurethane carrier composite immobilized substance according to claim 1, wherein the first mixture further comprises an auxiliary agent, and the auxiliary agent comprises a catalyst and/or a foam stabilizer.

9. The method for making a polyurethane carrier composite immobilized substance according to claim 8, wherein the catalyst comprises at least one of dimethylamino cyclohexane, dibutyltin dilaurate and bismuth octoate.

10. The method for making a polyurethane carrier composite immobilized substance according to claim 8, wherein a weight of the catalyst accounts for 100 ppm to 500 ppm of a weight of the second mixture.

11. The method for making a polyurethane carrier composite immobilized substance according to claim 8, wherein the foam stabilizer comprises a silicone surfactant.

12. The method for making a polyurethane carrier composite immobilized substance according to claim 8, wherein a weight of the foam stabilizer accounts for 0.1% to 10% of a weight of the second mixture.
